(19) United States
(12) Patent Application Publication
(10) Pub. No.: US 2009/0226511 A1
(43) Pub. Date: Sep. 10, 2009

(54) NOVEL PHARMACEUTICAL FORMULATION IN THE FORM OF CELLULOSE CAPSULES SUITABLE FOR BENZIMIDAZOLE DERIVATIVES

(75) Inventor: Judita SIRCA, Ljubljana (SI)

Correspondence Address: ARENT FOX LLP, 1050 CONNECTICUT AVENUE, N.W., SUITE 400, WASHINGTON, DC, 20036, US

(73) Assignee: Lek Pharmaceutical & Chemical Co. d.d., Ljubljana (SI)

(21) Appl. No.: 12/471,910

(22) Filed: May 26, 2009

Related U.S. Application Data
(63) Continuation of application No. 10/416,354, filed on May 9, 2003, now abandoned.

(30) Foreign Application Priority Data
Nov. 20, 2000 (SI) P-200000282

(51) Int. Cl. A61K 9/48; A61K 31/4439; A61P 1/00

(52) U.S. Cl. 424/452; 424/451; 514/338

(57) ABSTRACT

A filled cellulose capsule for oral administration, wherein the capsule comprises a cellulose derivative as the base and the filling comprises a therapeutically effective amount of a benzimidazole derivative but does not comprise a lower-substituted hydroxypropyl cellulose (13 to 16% hydroxypropoxy groups) is disclosed.

### TECHNICAL FIELD OF THE INVENTION

The invention belongs to the field of pharmaceutical technology and relates to the use of hard capsules for active substances that have poor water solubility, quickly disintegrate in an acidic medium and are unstable in the presence of moisture, solvents and acidic substances, whereas they have good stability in a neutral or alkaline medium, are quickly absorbed and metabolized and have an extended period of action. More precisely, the present invention relates to the use of hard capsules containing cellulose derivatives as the base, for the preparation of a pharmaceutical form with controlled release of active substances which are benzimidazole derivatives.

### TECHNICAL PROBLEM

There exists a constant need for the preparation of pharmaceutical formulations in the form of capsules, by which, in a technologically simple manner, a good stability of active substances that have poor water solubility, quickly disintegrate in an acidic medium and are unstable in the presence of moisture, solvents and acidic substances, whereas they have good stability in a neutral or an alkaline medium, are quickly absorbed and metabolized and have an extended period of action, is achieved. Hitherto known pharmaceutical formulations in the form of capsules containing such active substances use enteric coating pellets filled into hard gelatin capsules for providing a suitable stability of the active substance. These capsules have the disadvantage that they contain 13 to 15 wt. % of water. Therefore an additional drying of the capsules filled with enteric coating pellets is necessary.

### PRIOR ART

In U.S. Pat. No. 5,264,223 and U.S. Pat. No. 5,431,917 hard capsules containing water-soluble cellulose derivative as the base, a gel-forming agent and a gelling additive are described. An advantage of the described capsules is that they are less brittle in conditions of lower humidity.

In U.S. Pat. No. 5,756,123 hard gelatin capsules containing a water-soluble cellulose derivative such as hydroxypropylmethyl cellulose (HPMC) as the base are described. The main advantage of the described capsules is the use of the water-soluble cellulose derivative HPMC as the base, which does not disintegrate under special conditions (e.g. after consuming milk products).

In the article of T. Ogura, Y. Furuya, S. Matsuura (Pharmaceutical Technology Europe, November 1998, Volume 10, Number 11, pp. 32-42), a comparison between gelatin and HPMC capsules is described. It is stated that HPMC capsules are, inter alia, also suitable for active substances that are unstable in water. For active substances that are extremely sensitive to moisture, however, in order to increase their stability, pharmaceutically acceptable excipients absorbing water have to be added to the pharmaceutical formulation and an additional drying agent to the package nevertheless.

In WO 99/03453 there is described a novel pharmaceutical formulation with controlled release of active substances that are unstable in an acidic medium, unstable at extended storage in the presence of water and at the same time sensitive to heating, which is prepared by anhydrous granulation of active substances and additionally dried pharmaceutically acceptable substances with all pharmaceutically acceptable excipients dried prior to use in such a manner that their moisture content is lower than 1.0%, preferably lower than 0.5%. Enteric coating pellets are then filled into hard gelatin capsules.

### DESCRIPTION OF SOLUTION TO TECHNICAL PROBLEM WITH EXAMPLES

The invention is based upon the task to prepare a novel pharmaceutical formulation with active substances that have poor water solubility, quickly disintegrate in an acidic medium and are unstable in the presence of moisture, solvents and acidic substances, whereas they have good stability in a neutral or alkaline medium, are quickly absorbed and metabolized and have an extended period of action. In the narrow sense, the invention deals with a pharmaceutical formulation in which enteric coating pellets are filled into hard cellulose capsules consisting of a water-soluble cellulose derivative, preferably hydroxypropylmethyl cellulose (HPMC) as the base.

It has surprisingly been found that cellulose capsules are also suitable for active substances that have poor water solubility, quickly disintegrate in an acidic medium and are unstable in the presence of moisture, solvents and acidic substances, whereas they have good stability in a neutral or alkaline medium, are quickly absorbed and metabolized and have an extended period of action. It has to be pointed out that into the inventive pharmaceutical formulation as such, no pharmaceutically acceptable excipient absorbing water has to be added nor does any additional drying agent have to be inserted into the package.

In comparison to hard gelatin capsules, the technological process with cellulose capsules simplifies and shortens the technological process of manufacturing a pharmaceutical formulation according to the invention, since there is no need for any additional drying of already filled capsules to achieve the same stability of the active substances as by using hard gelatin capsules. The process of drying hard gelatin capsules lasts from 15 to 20 hours, which means that during all this time the active substance, which is poorly water-soluble and unstable in the presence of moisture, is exposed to the humid environment of the gelatin capsule.

By the use of cellulose capsules where drying is not necessary, the complete process of manufacturing the pharmaceutical formulation according to the invention is shortened, which is of essential importance with active substances that are poorly water-soluble, quickly disintegrate in an acidic medium and are unstable in the presence of moisture, solvents and acidic substances. Drying is an energy-consuming process, therefore the omission of dying saves large amounts of energy. Additionally, by the use of cellulose capsules instead of hitherto common hard gelatin capsules more production series of the final product can be made within the same period of time.

Pharmaceutical formulation according to the invention, wherein enteric coating pellets are filled into cellulose capsules, is especially suitable for active substances such as benzimidazole derivatives, which are known as substances that have poor water solubility, quickly disintegrate in an acidic medium and are unstable in the presence of moisture, solvents and acidic substances, whereas they have good stability in a neutral or alkaline medium, are quickly absorbed and metabolized and have an extended period of action. As active substances various benzimidazole derivatives can be used such as omeprazole, lansoprazole, timoprazole, rabeprazole, pantoprazole, leminoprazole, pariprazole, esomeprazole and their pharmaceutically acceptable salts such as sodium and magnesium salts. Active substances may also be in the form of their optical isomers and their pharmaceutically acceptable salts such as sodium and magnesium salts. These active substances are known as proton pump inhibitors and are used in the treatment of gastrointestinal diseases.

Enteric coating pellets to be filled in cellulose capsules are prepared according to a process of anhydrous granulation of active substances and of additionally dried pharmaceutically acceptable excipients, which are, prior to use, dried in such a manner that their moisture content is less than 1.0%, preferably less than 0.5%. The composition of enteric coating pellets and the manufacture thereof are described in WO 99/03453. Enteric coating pellets may be filled into cellulose capsules on all known capsulating machines that are usually used for filling pellets into hard gelatin capsules.

The invention is illustrated but in no way limited by the following working examples.

### EXAMPLE 1

### a) Pellet Cores

Composition for 1000 g of pellet cores.

| | | |
|---|---|---|
| Omeprazole | 100 | g |
| Low substituted hydroxypropylcellulose (13 to 16% of | 150 | g |
| hydroxypropoxy groups) | | |
| Microcrystalline cellulose | 150 | g |
| Mannitol | 478 | g |
| Sodium cross-linked carboxymethylcellulose | 50 | g |
| Polyvinylpyrrolidone K 25 | 70 | g |
| Polyoxyethylated hydrogenated castor oil | 2 | g |

The used pharmaceutically acceptable excipients were, prior to use, dried in such a manner that their moisture content was less than 1.0%, preferably less than 0.5%.

A series of 1000 g of pellet cores was prepared according to the following process:

2 g of polyoxyethylated hydrogenated castor oil (Cremophor® RH 40) was dissolved at room temperature in 300 g of absolute ethanol. At room temperature, the formed solution (302 g) was dispersed in a fluidized bed granulator onto previously prepared homogeneous mixture of powder components 100 g of omeprazole, 150 g of dried low-substituted hydroxypropylcellulose (L-HPC LH-20), 150 g of dried microcrystalline cellulose, 478 g of dried mannitol, 50 g of dried sodium cross-linked carboxymethylcellulose and 70 g of dried polyinylpyrrolidone K 25. So prepared plastic mixture was extruded and than spheronized. The formed pellet cores were dried in a fluidized bed or in a chamber dryer at a temperature of inlet air from 35 to 45° C., until the moisture content was less than 0.5%.

In such a manner 1000 g of pellet cores were obtained.

### b) Enteric Coating Pellets

| | | |
|---|---|---|
| Pellet cores | 1000 | g |
| Hydroxypropylmethyl cellulose phthalate | 150 | g |
| Dibutyl sebacate | 15 | g |

150 g of hydroxypropylmethyl cellulose phthalate and 15 g of dibutyl sebacate were dissolved at room temperature in a mixture of 1754 g of absolute ethanol and 438 g of acetone. The prepared solution was sprayed onto pellet cores in a fluidized bed apparatus.

### c) Capsulation

On a capsulating machine with gravimetrical filling the manufactured enteric coating pellets were filled into cellulose capsules Qualicaps Shionogi (cellulose capsules containing hydroxypropylmethyl cellulose as the cellulose derivative) with the content of omeprazole amounting to 20 mg/capsule.

### EXAMPLE 2

### a) Pellet Cores

Composition for 1000 g of pellet cores:

| | | |
|---|---|---|
| Lansoprazole | 100 | g |
| Microcrystalline cellulose | 200 | g |
| Mannitol | 598 | g |
| Sodium starch glycolate | 50 | g |
| Polyvinylpyrrolidone K 25 | 50 | g |
| Polysorbate 80 | 2 | g |

The used pharmaceutically acceptable excipients were, prior to use, dried in such a manner that their moisture content was less than 1.0%, preferably less than 0.5%.

Pellet cores were prepared according to the same process as in the Example 1 with exception that the active substance omeprazole was replaced by lansoprazole, dried low-substituted hydroxypropylcellulose (L-HPC LH-20) was replaced by microcrystalline cellulose, sodium cross-linked carboxymethylcellulose was replaced by sodium starch glycolate and the surfactant polyoxyethylated hydrogenated castor oil (Cremophor® RH 40) was replaced by Polysorbate 80.

### b) Enteric Coating Pellets

| | | |
|---|---|---|
| Pellet cores | 1000 | g |
| Eudragit L 100 | 150 | g |
| Dibutyl sebacate | 22 | g |
| Talc | 15 | g |

150 g of Eudragit and 22 g of dibtuyl sebacate were dissolved at room temperature in 1325 g of absolute ethanol and 15 g of talc were dispersed. Under constant stirring the prepared suspension was sprayed onto pellet cores in an apparatus with fluidized air.

### c) Capsulation

On a capsulating machine with gravimetrical filling the manufactured enteric coating pellets were filled into cellulose capsules Qualicaps Shionogi (cellulose capsules containing hydroxypropylmethyl cellulose as a cellulose derivative) with the content of lansoprazole amounting to 20 mg/capsule.

### EXAMPLE 3

Comparison of pharmaceutical formulations in the form of gelatin capsules and cellulose capsules and advantages of the use of cellulose capsules

### a) Preparation of Pharmaceutical Formulation

For the comparative test enteric coating pellets were prepared according to the process described in Example 1, points a) and b), whereupon, on a capsulating machine with gravimetrical filling, some were filled into hard gelatin capsules and others into cellulose capsules Qualicaps Shionogi (the cellulose derivative was hydroxypropyl-methyl cellulose).

### b) Drying of Pharmaceutical Formulation

In the technological process of manufacturing the pharmaceutical formulation this phase took place immediately after capsulating.

TABLE 1
Comparison of drying time of capsules in the technological
process of manufacturing the pharmaceutical formulation

| | Pharmaceutical formulation | Pharmaceutical formulation |
|---|---|---|
| Capsules used | using hard gelatin capsules | using cellulose capsules |
| Drying time | 15-20 hours | 0 hours |

### c) Stability of Active Substance

TABLE 2
Comparison of stability of pharmaceutical forms

| | Pharmaceutical | Pharmaceutical |
|---|---|---|
| | formulation | formulation |
| | using hard | using cellulose |
| Capsules used | gelatin capsules | capsules |
| Related substances and | 1.10% | 1.09% |
| disintegration products | | |
| after 3 months at conditions | | |
| of 40° C. and 75% relative | | |
| humidity | | |

Determination of Related Substances and Disintegration Products

| Principle | |
|---|---|
| Chromatography conditions | HPLC determination |
| stationary phase | Symmetry C8 |
| dimensions: | 150 × 4.6 mm |
| temperature: | 30° C. or controlled room temperature, |
| Mobile phase: | Buffer:acetonitrile |
| | 300:100, vol. ratio, |
| Flow: | 0.8 ml/min, |
| Injection volume | 50 μl |
| Wave length | UV, 320 nm. |

From the above tables it is evident that in comparison with the hard gelatin capsules the use of cellulose capsules does not impair the stability of the active substance, yet it brings big savings in the technological process due to the omission of the long drying phase (energy savings, larger production capacity).

A great advantage of the use of cellulose capsules is also the fact that the active substance that is unstable in the presence of moisture is not exposed to the humid environment of the gelatin capsule for a lengthy period of time.

Into the pharmaceutical formulation according to the invention that is manufactured according to the process of anhydrous granulation of the active substance and of dried pharmaceutically acceptable excipients, no pharmaceutically acceptable water-absorbing excipient has to be added. Due to the smaller number of excipients the novel pharmaceutical formulation puts less strain on the patient's organism.

No drying agent has to be added into the final package.

The capsulating of the pharmaceutical formulation with cellulose capsules according to the invention is performed on the same equipment i.e. the same capsulating machines as with hard gelatin capsules.

1-37. (canceled)

38. A filled cellulose capsule for oral administration, wherein the capsule comprises a cellulose derivative as the base and the filling comprises a therapeutically effective amount of a benzimidazole derivative but does not comprise a lower-substituted hydroxypropyl cellulose (13 to 16% hydroxypropoxy groups).

39. The filled capsule of claim 38 where the cellulose derivative of the capsule is hydroxypropylmethyl cellulose.

40. The filled capsule of claim 38 where the benzimidazole derivative is selected from the group consisting of omeprazole, lansoprazole, timoprazole, rabeprazole, pantoprazole, leminoprazole, pariprazole, esomeprazole, their pharmaceutically acceptable salts, their optically active isomers, and pharmaceutically acceptable salts of their optically active isomers.

41. The filled capsule of claim 40 where the benzimidazole derivative is omeprazole or a pharmaceutically acceptable omeprazole salt.

42. The filled capsule of claim 40 where the benzimidazole derivative is esomeprazole or a pharmaceutically acceptable esomeprazole salt.

43. The filled capsule of claim 40 where the benzimidazole derivative is lansoprazole or a pharmaceutically acceptable lansoprazole salt.

44. The filled capsule of claim 40 where the benzimidazole derivative is an optically active isomer of lansoprazole or a pharmaceutically acceptable salt of an optically active isomer of lansoprazole.

45. The filled capsule of claim 38 where the filling comprises enterically coated pellets prepared by anhydrous granulation of the benzimidazole derivative and a dried pharmaceutically acceptable excipient.

46. The filled capsule of claim 45 where the dried pharmaceutically acceptable excipient comprises microcrystalline cellulose.

47. The filled capsule of claim 45 where the benzimidazole derivative is selected from the group consisting of omeprazole, lansoprazole, timoprazole, rabeprazole, pantoprazole, leminoprazole, pariprazole, esomeprazole, their pharmaceutically acceptable salts, their optically active isomers, and pharmaceutically acceptable salts of their optically active isomers.

48. The filled capsule of claim 47 where the benzimidazole derivative is omeprazole or a pharmaceutically acceptable omeprazole salt.

49. The filled capsule of claim 47 where the benzimidazole derivative is esomeprazole or a pharmaceutically acceptable esomeprazole salt.

50. The filled capsule of claim 47 where the benzimidazole derivative is lansoprazole or a pharmaceutically acceptable lansoprazole salt.

51. The filled capsule of claim 47 where the benzimidazole derivative is an optically active isomer of lansoprazole or a pharmaceutically acceptable salt of an optically active isomer of lansoprazole.

52. The filled capsule of claim 45 where each pharmaceutically acceptable excipient in the pellets has been dried to a moisture content of less than 1% before granulation.

53. The filled capsule of claim 52 where each pharmaceutically acceptable excipient in the pellets has been dried to a moisture content of less than 0.5% before granulation.

54. A process for the preparation of a filled cellulose capsule, the process comprising preparing by anhydrous granulation enteric coated pellets comprising a therapeutically effective amount of a benzimidazole derivative and dried pharmaceutically acceptable excipients, and filling a hydroxypropylmethyl cellulose based capsule with the enterically coated pellets, wherein said pellets do not comprise lower-substituted hydroxypropyl cellulose (13-16% hydroxypropoxy groups) and wherein said process does not comprise further drying of said filled capsules.

55. The process of claim 54 wherein said pellets comprises microcrystalline cellulose.

56. A method of treatment of a gastrointestinal disease in a patient suffering from the gastrointestinal disease, comprising administering to the patient a filled capsule of claim 45.

57. The method of claim 56 where the cellulose derivative is hydroxypropylmethyl cellulose.

58. The method of claim 56 where the benzimidazole derivative is selected from the group consisting of omeprazole, lansoprazole, timoprazole, rabeprazole, pantoprazole, leminoprazole, pariprazole, esomeprazole, their pharmaceutically acceptable salts, their optically active isomers, and pharmaceutically acceptable salts of their optically active isomers.

59. The method of claim 58 where the benzimidazole derivative is omeprazole or a pharmaceutically acceptable omeprazole salt.

60. The method of claim 58 where the benzimidazole derivative is esomeprazole or a pharmaceutically acceptable esomeprazole salt.

61. The method of claim 58 where the benzimidazole derivative is lansoprazole or a pharmaceutically acceptable lansoprazole salt.

62. The method of claim 58 where the benzimidazole derivative is an optically active isomer of lansoprazole or a pharmaceutically acceptable salt of an optically active isomer of lansoprazole.

63. The method of claim 56 where each pharmaceutically acceptable excipient in the pellets has been dried to a moisture content of less than 1% before granulation.

64. The method of claim 56 where each pharmaceutically acceptable excipient in the pellets has been dried to a moisture content of less than 0.5% before granulation.
